(19) United States
(12) Patent Application Publication
(10) Pub. No.: US 2026/0306046 A1
(43) Pub. Date: Oct. 8, 2026

(54) DEVICE

(71) Applicant: CAMBRIDGE ENTERPRISE LIMITED, Cambridge (GB)

(72) Inventors: Andrew MCDONALD, Cambridgeshire (GB); Anurag AGARWAL, Cambridgeshire (GB); Maximilian NUSSBAUMER, Cambridgeshire (GB)

(73) Assignee: CAMBRIDGE ENTERPRISE LIMITED, Cambridge (GB)

(21) Appl. No.: 19/481,507

(22) PCT Filed: May 3, 2024
(86) PCT No.: PCT/GB2024/051172

§ 371 Date: Nov. 4, 2025

(30) Foreign Application Priority Data
May 5, 2023 (GB) 2306681.4

(51) Int. Cl. A61B 7/04; A61B 5/00; A61B 5/11; A61B 7/00

(52) U.S. Cl. CPC A61B 7/04; A61B 5/1126; A61B 5/6823; A61B 5/6833; A61B 5/7221; A61B 5/7264; A61B 7/003; A61B 7/008; A61B 5/743; A61B 2562/046; A61B 2562/164

(57) ABSTRACT

According to a first aspect of the disclosure there is provided a data collection device for monitoring a physiological state of a subject, comprising: a plurality of sensors, each of the plurality of sensors being configured to obtain a biophysical signal from the subject; a flexible substrate configured to be placed against the subject by a user and configured to flex to substantially conform with the form of the subject; wherein the plurality of sensors are connected to the substrate and arranged such that each sensor is configured to move relative to each other sensor to enable the substrate to flex to substantially conform to the form of the subject.

### TECHNICAL FIELD

The present disclosure relates to devices, systems and methods for monitoring the physiological state of a subject. In an example, vibrational signals from the heart of a subject may be monitored.

### BACKGROUND ART

Typically, valvular heart disease is detected in subjects by a trained clinician listening to heart sounds using a stethoscope. However, this relies on human detection of abnormal heart sounds, which means the assessment has an element of subjectivity. Further, this requires highly experienced clinicians having undergone significant training, which limits the number of assessments that can be carried out. Detection of other diseases, including diseases of the heart, lungs, bowel or throat may be performed in a similar manner.

Electronic stethoscope devices are known in the art. However, these generally require accurate placement on the subject, which is difficult to achieve without specialist training.

It is an aim of the present disclosure to at least partially address some of the above problems.

### SUMMARY OF THE INVENTION

According to a first aspect of the disclosure there is provided a data collection device for monitoring the physiological state of a subject, comprising: a plurality of sensors, each of the plurality of sensors being configured to obtain a biophysical signal from the subject; a flexible substrate configured to be placed against the subject by a user and configured to flex to substantially conform with the form of the subject; wherein the plurality of sensors are connected to the substrate and arranged such that each sensor is configured to move relative to each other sensor to enable the substrate to flex to substantially conform to the form of the subject. Such a device may provide relatively high quality data for monitoring the physiological state of a subject, while being relatively easy to use.

Optionally, the flexible substrate is arranged between the plurality of sensors and the subject, in use.

Optionally, the device is a handheld device.

Optionally, the device has a maximum width of no more than 15 cm.

Optionally, the plurality of sensors are arranged in two dimensions.

Optionally, the plurality of sensors comprises a central sensor surrounded by a plurality of further sensors.

Optionally, the plurality of sensors are arranged in an arrangement that has rotational symmetry.

Optionally, adjacent sensors of the plurality of sensors are arranged at least 2 cm apart.

Optionally, adjacent sensors of the plurality of sensors are arranged at least 2.5r apart, where r is the radius of the sensor, or half the width of the sensor at its widest point.

Optionally, adjacent sensors of the plurality of sensors are arranged no more than 5 cm apart.

Optionally, sensors of the plurality of sensors that are furthest apart are arranged at least 5 cm apart.

Optionally, sensors of the plurality of sensors that are furthest apart are arranged no more than 15 cm apart.

Optionally, the device is configured to achieve a radius of curvature of less than 40 cm when flexed.

Optionally, the device is configured to achieve a radius of curvature of less than 30 cm when flexed.

Optionally, the plurality of sensors are substantially spread across the flexible substrate.

Optionally, the flexible substrate is substantially circular.

Optionally, an outer surface of the flexible substrate that is configured to contact the subject in use, is substantially smooth.

Optionally, an outer surface of the flexible substrate that is configured to contact the subject in use, is configured to be wiped clean.

Optionally, the flexible substrate is formed from an elastomeric material. Optionally, the flexible substrate is formed from silicone.

Optionally, each of the plurality of sensors is connected to the flexible substrate by an adhesive.

Optionally, each of the plurality of sensors is retained within moulded portions of the flexible substrate.

Optionally, the plurality of sensors are connected to each other by flexible connectors.

Optionally, each sensor of the plurality of sensors is configured to obtain a vibration signal.

Optionally, each sensor of the plurality of sensors is the same.

Optionally, the biophysical signal is a vibration signal from the heart of the subject.

Optionally, each of the plurality of sensors is configured to obtain a signal simultaneously.

Optionally, the device further comprises a communication unit configured to communicate the biophysical signals obtained by the plurality of sensors to an external device for processing. Optionally, the communication unit is configured to communicate wirelessly with the external device. Optionally, the communication unit is configured to receive instructions from the external device for controlling the plurality of sensors.

According to a second aspect of the disclosure there is provided a system for monitoring the physiological state of a subject, the system comprising: the device of the first aspect; one or more processors configured to process the biophysical signals obtained by the plurality of sensors in order to determine the physiological state of a subject.

Optionally, the one or more processors is configured to base the determination of the physiological state of a subject on a subset of the biophysical signals obtained by the sensors.

Optionally, the subset of biophysical signals comprises at least one signal that is of a relatively high quality compared to other biophysical signals not in the subset.

Optionally, the one or more processors are configured to determine the physiological state of a subject based on biophysical signals obtained when the device is placed against the subject at at least two different predefined locations.

Optionally, the one or more processors form part of the external device optionally in communication with the device of the first aspect.

According to a third aspect of the disclosure there is provided a system for monitoring a physiological state of a subject, comprising: a data collection device comprising a plurality of sensors, each of the plurality of sensors being configured to obtain a biophysical signal from the subject; one or more processors configured to process signals obtained by the plurality of sensors to determine the quality of the signals, and store a subset of the signals for further processing in order to determine the physiological state of a subject. Such a system may allow relatively high quality data for monitoring the physiological state to be obtained, using a device that is relatively easy to use.

Optionally, the one or more processors are further configured to process the stored subset of the signals to determine the physiological state of a subject.

According to a fourth aspect of the disclosure, there is provided a system for monitoring a physiological state of a subject, comprising: a data collection device configured to be placed on a subject, and comprising a plurality of sensors, each of the plurality of sensors being configured to obtain a biophysical signal from the subject; one or more processors configured to process signals obtained by the plurality of sensors to determine whether the data collection device is correctly placed on the subject. Such a system may allow relatively high quality data for monitoring the physiological state to be obtained, using a device that is relatively easy to use.

Optionally, the determination of whether the data collection device is correctly placed on the subject is based on whether one or more signals include a useful biophysical signal, that is a signal based on which the physiological state of the subject can be determined.

According to a fifth aspect of the disclosure there is provided a computer implemented method of monitoring the physiological state of a subject, the system comprising: receiving data including one or more biophysical signals obtained by the device of the first aspect; and processing the data including the one or more biophysical signals to determine the physiological state of a subject.

According to a sixth aspect of the disclosure there is provided a data collection method of collecting data for monitoring the physiological state of a subject, comprising: providing a device comprising: a plurality of sensors, each of the plurality of sensors being configured to obtain a biophysical signal from the subject; a flexible substrate configured to be placed against the subject by a user and configured to flex to substantially conform with the form of the subject, the flexible substrate being arranged between the plurality of sensors and the subject, in use; wherein the plurality of sensors are connected to the substrate and arranged such that each sensor is configured to move relative to each other sensor to enable the substrate to flex to substantially conform to the form of the subject; placing the device on the subject; and obtaining a biophysical signal from the subject using the device. Such a method may provide relatively high quality data for monitoring the physiological state of a subject, while being relatively easy to perform. There may alternatively be provided a method of collecting data for monitoring a physiological state of a subject, the method comprising use of the data collection device of the first aspect to obtain a biophysical signal from a subject.

Optionally, the methods comprise placing the flexible substrate of the device against the subject at at least one location on the subject to obtain the biophysical data. Optionally, the at least one location corresponds a location suitable for obtaining a biophysical data corresponding to at least one predetermined part of the subject. Optionally, the part is the heart. Optionally, the part is at least one of the tricuspid valve and the mitral valve of the heart. Alternatively, the part of the subject is the lungs, bowel or throat.

### BRIEF DESCRIPTION OF THE DRAWINGS

Further features of the disclosure will be described below, by way of non-limiting examples and with reference to the accompanying drawings, in which:

FIG. 1 shows an example device according to the disclosure;

FIG. 2 shows an exploded view of the example device;

FIG. 3 shows electronic components of the example device;

FIG. 4 shows the device in use, recording data relating to valvular heart disease;

FIG. 5 shows an external device (a smartphone) being used with the device; and

FIG. 6 shows example data obtained by sensors of the device;

FIG. 7 shows further example data obtained by three different types of sensors;

FIG. 8 is a table showing signal-to-noise ratio for example data obtained by three different types of sensors;

FIG. 9 schematically shows an example arrangement of sensors on a substrate.

### DETAILED DESCRIPTION

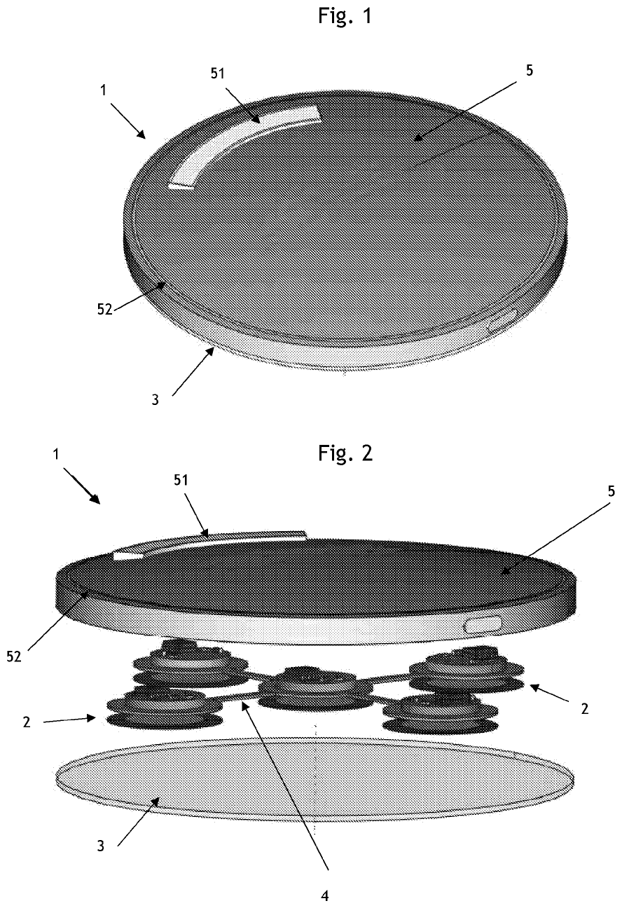

FIG. 1 shows an example device 1 according to the disclosure and FIG. 2 shows an exploded view of the device 1. As shown in FIG. 2, the device 1 comprises a plurality of sensors 2. As shown, the device 1 may further comprise a flexible substrate 3. As shown, the device may further comprise a casing 5 covering the sensors 2.

The plurality of sensors 2 are configured to obtain a biophysical signal from the subject. For example, the sensors 2 may be configured to obtain a vibration signal from the heart of the subject, as in the example device 1 shown. However, alternatively or additionally, the sensors 2 may be configured to obtain different biophysical signals, such as acoustic signals from other organs, such as the lungs, bowel or throat. Alternatively, electrical signals may be obtained. In some examples, different sets of sensors 2 may be combined in a single device, each set comprising a different type of sensor, e.g. sets of vibration and electrical sensors. In such examples, the plurality of sensors 2 as a whole may have the features described below and/or each set of sensors 2 separately.

The sensors 2 may comprise sensors that are configured to detect vibrations from the subject. For example, the sensors 2 may comprise a piezoelectric transducer, an accelerometer (e.g. MEMS accelerometer), or a microphone (e.g. air-coupled).

The flexible substrate 3 is configured to be placed against the subject by a user of the device (which may be the subject themselves, or a different person) and is arranged between the plurality of sensors 2 and the subject, in use. The flexible substrate 3 is further configured to flex to substantially conform with the form of the subject, where it is placed. The flexible substrate 3 may be formed from an elastomeric material, for example, e.g. silicone.

An outer surface of the flexible substrate 3 that is configured to contact the subject in use, may be substantially smooth. This may help to prevent damping of the signals detected by the sensors from the subject. The outer surface of the flexible substrate 3 that is configured to contact the subject in use, may be configured to be wiped clean. This may allow the device to be easily reused for different subjects. The flexible substrate 3 may be formed from silicone having a smooth outer surface, for example.

The plurality of sensors 2 may be connected to the substrate 3. The connection may be such that the sensors 2 can move together with the portion of the substrate 3 to which they are connected. The sensors 2 may be arranged such that each sensor 2 is configured to move relative to each other sensor 2. For example, the sensors 2 may independently move together with the portion of the substrate 3 to which they are connected. The sensors 2 may be arranged to enable the substrate 3 to flex to substantially conform the form of the subject.

As shown in FIG. 2, the sensors 2 may be connected to the substrate by an adhesive. This may be in the form of an adhesive layer or patch. One adhesive layer or patch may be provided for each sensor 2, as shown. Alternatively, or additionally, the sensors 2 may be connected to the substrate 3 by means of their retention within moulded portions of the substrate. In other words, the flexible substrate 3 may comprise moulded portions having openings configured to retain the sensors 2 therein. The moulded portions may be flexible.

As shown in FIG. 2, the sensors 2 may be connected to each other. As shown in FIG. 2, this connection may be provided by one or more flexible connectors 4. The one or more flexible connectors 4 may be configured to allow the sensors 2 to move relative to each other. As in the example shown in FIG. 2, the connectors may comprise electrical wiring. For example, the connectors 4 may be flexible ribbon cables.

In some examples, flexible connectors 4 may be configured to allow the sensors 2 to move relative to each other, while maintaining the relative positions of the sensors 2 in a neutral, non-flexed arrangement. In the example shown a single connector may be provided that connects each of the plurality of sensors 2 by flexible arms. The connector 4 may comprise attachment portions, for attaching to each of the sensors 2, connected by the flexible arms. For example, such flexible connectors may be formed from an elastomeric material, e.g. silicone.

As shown in FIG. 1, the outer casing 5 may be configured to cover the sensors 2 and other components of the device (such as the electronic components described below). The casing 5 is provided on the opposite side of the sensors 2 to the substrate 3. The outer casing 5 may be substantially concave, the cavity formed by the outer casing being configured to house the device components, including the sensors 2. An opening in the outer casing 2 may be covered by the flexible substrate 3.

The outer casing 5 may enable the substrate 3 to flex to substantially conform with the form of the subject. For example, the outer casing 5 may itself be flexible. For example, the outer casing 5 may be formed from an elastomer material, such as silicone.

As shown, the outer casing 5, may further comprise a button 51 (e.g. power button) for controlling the device and a light source 52 (e.g. LEDs) to enable communication between the device and the user of the device.

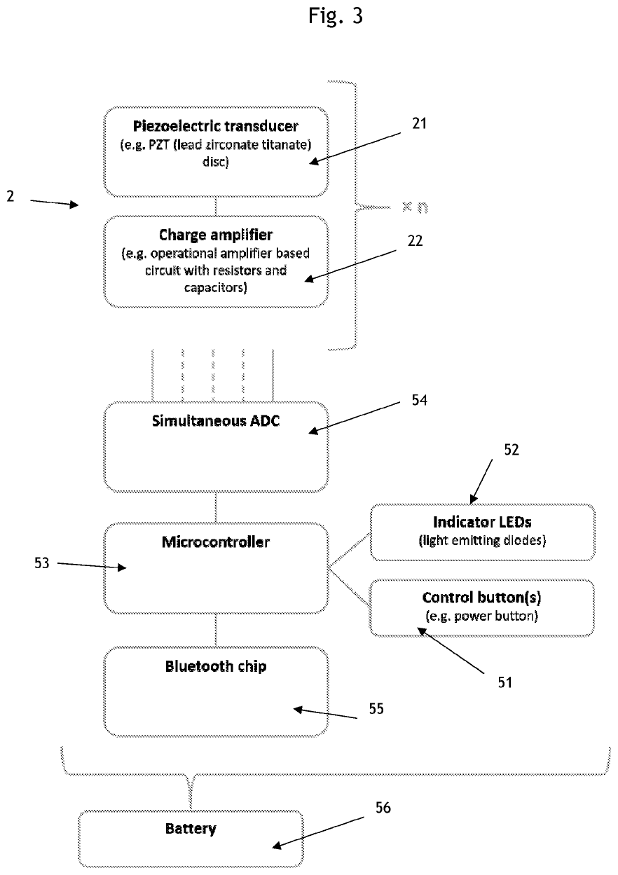

FIG. 3 shows electronic components of the example device 1. As described above, these components may be housed within the outer casing 5. As shown in FIG. 3, the button 51 and the light source 52 may be connected to a processor 53 of the device 1, such as a microcontroller. As shown, the processor 53 may be additionally connected to the sensors 2, e.g. via one or more analogue-to-digital converters. Signals obtained by the sensors 2 may be communicated to the processor 53.

As shown in FIG. 3, the device 1 may comprise a communication unit 55, such as a Bluetooth™ chip. The communication unit 55 may be configured to communicate the signals obtained by the plurality of sensors 2 to an external device, e.g. for processing. The communication unit 55 may be configured to communicate wirelessly with the external device. As shown, the communication unit 55 may be connected to the processor 53 of the device 1. The processor 53 may control the communication unit 55.

Alternatively, or additionally, the communication unit 55 may be configured to receive instructions from an external device for controlling the device 1, e.g. the plurality of sensors 2. These instructions may be provided by the communication unit 55 to the processor 53. The processor 53 may control the other components of the device 1, such as the sensors 2, based on the received instructions.

As shown, in this example device, the sensors 2 may comprise a piezoelectric transducer 21 and a charge amplifier 22. As discussed above, different types of sensors 2 may alternatively be used.

As shown in FIG. 3, the device 1 may comprise a power source 56, such as a battery, for providing power to the device 1.

The device 1 may have a width (parallel to the substrate 3) of no more than around 15 cm, at its widest point, for example. Sensors of the plurality of sensors that are furthest apart may be arranged no more than 15 cm apart, for example. A device 1 having such features may be easy for a user to position on the subject. The device 1 may be a handheld device. For example, a user may place the device 1 on the subject by hand.

Each of the plurality of sensors 2 may be configured to obtain the same type of biophysical signal. For example, each of the sensors 2 may be substantially the same. This may ensure that a useful biophysical signal is obtained even when some (or all but one) of the sensors 2 are incorrectly placed on the subject. In other words, a useful signal may still be obtained if only one of the sensors is correctly placed. Providing a plurality of sensors 2 may increase the likelihood of at least one of the sensors being correctly placed.

At least three sensors 2 may be provided. As shown in FIG. 2, five sensors 2 may be provided, for example. A higher number of sensors 2 may advantageously increase the chance that at least one sensor 2 is correctly placed to obtain a useful biophysical signal.

As shown in FIG. 3, the plurality of sensors 2 may be arranged in two-dimensions, e.g. in a two-dimensional array. The sensors 2 may be arranged to be spread out over a sensing area of the device. The sensing area may correspond to at least a portion of the flexible substrate 3. As in the example device shown in FIG. 3, the sensing area may cover a substantial portion of the flexible substrate 3, or substantially all of the flexible substrate 3. The plurality of sensors 2 may be substantially spread across the flexible substrate 3.

Sensors of the plurality of sensors 2 that are furthest apart may be arranged at least 5 cm apart, for example. The device 1 may have a width (parallel to the substrate 3) of at least 5 cm, at its widest point, for example. Spreading out the plurality of sensors 2 may increase the likelihood of at least one of the sensors 2 being correctly placed. Each sensor 2 may be arranged no more than 5 cm from another sensor 2, e.g. as measured centre to centre. This may reduce the likelihood of signal blind spots, and/or may increase the likelihood of at least two sensors obtaining relatively high quality signals, which may enable the output to be improved by corroborating signals from multiple sensors.

The sensors 2 may be arranged such that adjacent sensors are no more than 5 cm apart, centre to centre, preferably no more than 3 cm apart, optionally no more than 2 cm apart. If the sensors 2 are arranged too far apart, this may reduce the likelihood that at least one sensor 2 is correctly placed on the subject to obtain a useful biophysical signal.

As shown in FIG. 3, the plurality of sensors 2 may be arranged to have a central sensor 2 surrounded by a plurality of further sensors 2. As shown, one central sensor 2 may be surrounded by four further sensors 2. The plurality of sensors 2 may be arranged in concentric groups, for example. Each group may comprise a plurality of sensors 2, with one group surrounding another group.

As shown, the plurality of sensors 2 may be arranged to have bilateral symmetry. As shown, the plurality of sensors 2 may be arranged to have rotational symmetry. The order of rotational symmetry may be at least three. This may be achieved with three sensors arranged in a triangular arrangement, optionally with a central sensor. As shown in FIG. 3, the order of rotation symmetry may be four, with the outer sensors may be arranged in square. Alternatively, the sensors 2 may be arranged in any other regular shape. Different concentric groups of sensors 2 may have different orders of rotational symmetry. For example, the sensors 2 may include a triangularly arranged inner group surrounded by an outer group arranged in a square.

Providing a flexible substrate 3 that can substantially conform to the form of the subject, and plurality of sensors 2 that can move relative to each other sensor to enable the substrate to flex to substantially conform to the form of the subject, may increase the likelihood that at least one sensor 2 is correctly placed on the subject to obtain a useful biophysical signal.

The sensors 2 may be arranged such that adjacent sensors 2 are at least 2 cm apart, centre to centre. The sensors 2 may be arranged to have a gap between adjacent sensors 2 of at least 1 cm, for example, preferably at least 1.5 cm. If the sensors 2 are arranged too close together may reduce the ability of the flexible substrate 3 to flex to conform to the form of the subject.

The sensors 2 may be arranged such that adjacent sensors 2 are arranged spaced apart, centre to centre, by at least 2.5 times the sensor radius, or 2.5 times half the width at the widest point of the sensor 2. The sensors 2 may be substantially circular in shape.

The flexible substrate 3 may be configured to achieve a radius of curvature of less than 40 cm when flexed, preferably less than 30 cm. Accordingly, the flexible substrate is able to conform to most required body areas of subjects.

FIG. 9 shows schematically shows a further example arrangement of sensors 2 on the substrate 3. As shown, five sensors may be arranged at the points of a regular pentagon, with a central sensor arranged at the centre of the pentagon. The sensors 2 at the points of the pentagon may be separated by around 4 cm, centre to centre. The sensors may have a diameter of around 2 cm, i.e. 1.8 cm as shown. As shown, the flexible substrate 3 may be circular, and may have a diameter of around 10 cm. The central sensor 2 may be arranged in the centre of the substrate 3.

The device 1 may be a handheld device, as discussed above. However, the device 1 may also be incorporated into a garment to be worn by a subject, such as a strap. The device 1 may comprise an adhesive layer on the outer surface of the substrate so the device 1 can stick to the subject. The adhesive may be reusable so the device 1 can be relocated.

The device 1 may be configured such that useful biophysical signals may be obtained over the clothing of a subject (e.g. having a thickness no more than a predefined thickness), so that direct contact with the subject's skin is not required.

A device according to the disclosure, such as the example device 1 described above in relation to FIGS. 1 to 3, may be used to obtain data relating to one or more biophysical signals from a subject, for monitoring the subject. The device may therefore be regarded as a data collection device. The data collected by the device may be processed to determine the physiological state, or health, of the subject. Processing may be performed by one or more processors forming part of an external device, part of the data collecting device itself, or a combination of both.

The one or more sensors may obtain signals that may be biophysical signals or non-biophysical signals. A biophysical signal obtained by a sensor may be a signal that includes information relating to a biophysical signal from the subject, such as vibrations generated by the heart, lungs or bowel. Such a biophysical signal obtained by a sensor may have specific characteristics identifying the signal as including information relating to a biophysical signal from the subject.

A biophysical signal may be defined as a signal that includes information relating to a specific type of biophysical signal from the subject. The type of biophysical signal may be determined by the intended use of the device, e.g. biophysical signals that allow the physiological state of the subject to be determined. These biophysical signals may also be referred to as useful biophysical signals. For example, a biophysical signal including information relating to vibrations generated by the heart of the subject, may be considered a useful biophysical signal if the intended use is to monitor the heart, but a signal including only information relating to the vibrations generated by the lungs may not be considered a biophysical signal if the intended use is to monitor the heart. A useful biological signal may be obtained when the data collection device is correctly placed on the subject.

The data collection device may be configured such that each of the plurality of sensors obtains a signal from the subject when the device is placed at the same location and at substantially the same time. The signals are preferably obtained simultaneously. However, the signals may alternatively be obtained sequentially.

The quality of the obtained signals may vary depending on the specific location of each sensor on the subject, and the placement of the device. For example, some sensors may be obstructed by physiological structures of the subject, such as the ribs, or clothing, and/or a gap may be formed between the device and the subject at the location of the sensors. Other sensors may be unobstructed and/or the device may be in close contact with the subject at the location of the sensors.

The one or more processors may be configured to determine a quality level for each signal obtained by the sensors. Based on the quality level, one or more signals having a relatively high quality may be processed further to determine the physiological state, or health, of the subject. For example, the one highest quality signal may be processed further. The quality may be determined based on a signal to noise ratio of the signal. This may be determined based on the relative strength of signal characteristics expected of a high quality useful biophysical signal. This processing may be performed on the external device, or the data collecting device. A high quality signal may have a signal-to-noise ratio of at least 5 dB, for example.

The one or more processors may be configured to determine, based on the signals obtained by the sensors (e.g. the one or more relatively high quality signals), whether the signals are indicative of a normal or an abnormal physiological state. The one or more processors may output data indicating the likelihood of the subject having a normal or an abnormal physiological state. The processors may output data relating to the likelihood of the signals being indicative of one or more specific abnormal physiological states, or health conditions. This processing may preferably be performed by an external device.

The one or more processors may be configured to execute a machine learning algorithm trained to classify the one or more signals as indicative of a normal or an abnormal physiological state, or indicative of one or more specific abnormal physiological states, or health conditions.

Only some of the data obtained by the data collection device may be processed to determine the physiological state of the subject. This may be data obtained under specific conditions, for example when the device is placed correctly on the subject. Otherwise, data collected may not be processed to determine the physiological state of the subject. However, data obtained when the device is not placed correctly on the subject may still be used for a different purpose.

The data collection device may be configured to automatically (e.g. without user input) provide data to the one or more processors to be processed to determine the physiological state of the subject, only when the data corresponds to the device being correctly positioned on the subject. Alternatively, data may be provided by the data collection device to the one or more processors, but only processed to determine the physiological state of the subject when the data corresponds to the device being correctly positioned on the subject.

The data collection device may be configured to continuously, or periodically, obtain data. The one or more processors, may be configured to determine, based on the data, whether the data includes a useful biophysical signal. If a useful biophysical signal is determined to be present, then the one or more processors may determine that the device is correctly placed on the subject. Such data may then be processed to determine the physiological state of the subject. If a useful biophysical signal is not detected based on the obtained data, the obtained data may not be processed to determine the physiological state of the subject. Such data may be discarded, for example. A useful biophysical signal may be required to be identified in at least one signal obtained from at least one respective sensor. For example, only one useful biophysical signal from the plurality of signals may be required.

The data collection device may be configured to automatically stop providing data to be processed to determine the physiological state of the subject, when a useful biological signal is obtained that allows a determination of the physiological state of the subject. For example, this may be after useful biophysical signals of a predetermined length of time are obtained.

In certain use cases, it may be required that useful biological signals are collected when the data collection device is positioned at a plurality of different locations on the subject. Accordingly, the device may be configured to collect first data to be processed to determine the physiological state of the subject, then stop collecting data to be processed to determine the physiological state of the subject, then restart collecting data to be processed to determine the physiological state of the subject when the device is correctly relocated.

The relocation of the device may be determined based on data obtained by the sensors. For example, after data collection at the first location, removal of the device from the subject may be determined, e.g. when a useful biophysical signal is no longer identified in the data. When a useful biophysical signal is identified in the data again, collecting data to be processed to determine the physiological state of the subject may be restarted.

Data to be processed to determine the physiological state of the subject may be stored in a memory associated with the one or more processors. For example, all data collected may be stored in a first memory, then data to be processed to determine the physiological state of the subject may be transferred to a second memory. Data in the first memory not transferred to the second memory may be discarded or overwritten without further processing to determine the physiological state of the subject.

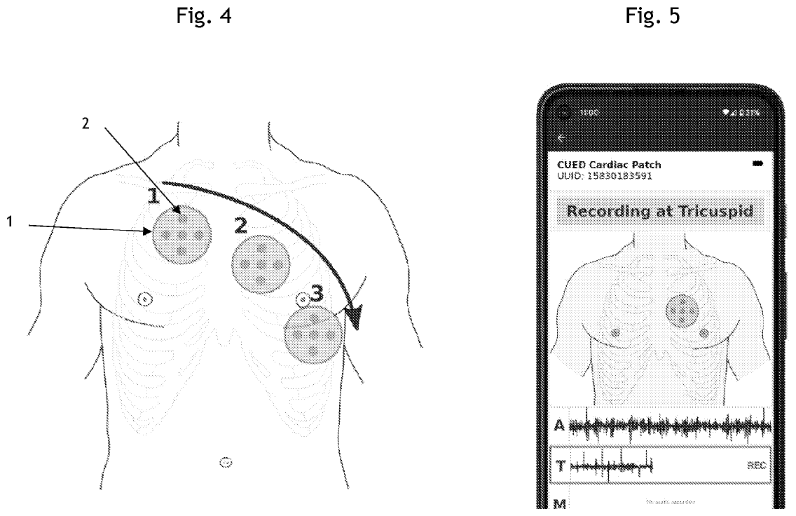

FIGS. 4 and 5 show a first example system comprising a data collection device according to the disclosure and an external device. In the example use case shown, biophysical signals are obtained when the data collection device 1 is placed at three different locations, namely locations 1-3 in FIG. 4. In the examples, these locations correspond to locations suitable for obtaining signals corresponding to the aortic, tricuspid and mitral heart valves respectively.

As shown in FIG. 5, the external device may be a smartphone. However, alternatively, the external device may be a personal computer or tablet computer. The external device may be configured to store and process data obtained by the data collection device 1. For example, the external device may be configured to store the data to be processed to determine the physiological state of the subject. For example, the external device may comprise the second memory described above.

The external device may be configured to provide the user with instructions regarding the placement of the data collection device. As shown in FIG. 5, these instructions may be visual instructions showing an image of the location at which the data collection device should be placed. Alternatively, or additionally, written and/or audible instructions may be provided to the user by the external device. As shown in FIG. 5, the device may additionally provide the user with information relating to the biophysical signals obtained by the device, e.g. showing these visually as they are obtained and stored.

Information may also be provided to the user by the data collection device itself. For example, correct placement of the device may be indicated by the light source 52 on the device. When data to be processed to determine the physiological state of the subject is collected by the device, this may be indicated by the light source 52.

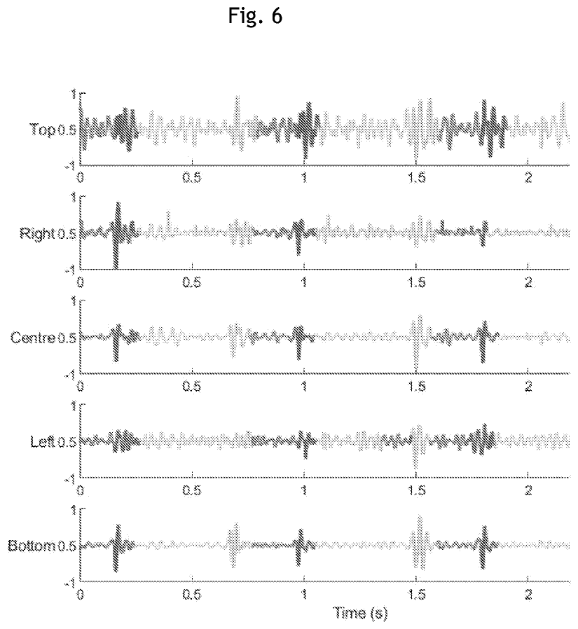

FIG. 6 shows illustrative signals obtained by the five sensors 2 of the device 1. Signal characteristics of the biophysical signal are shown in darker shades. These correspond to heart valve sounds in this example. As shown, the signals are of varying quality, with the highest quality signal corresponding to the bottom signal.

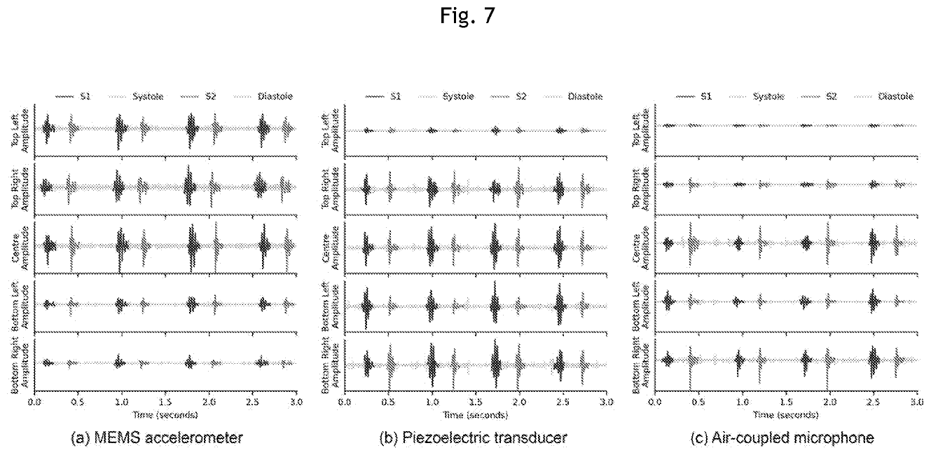

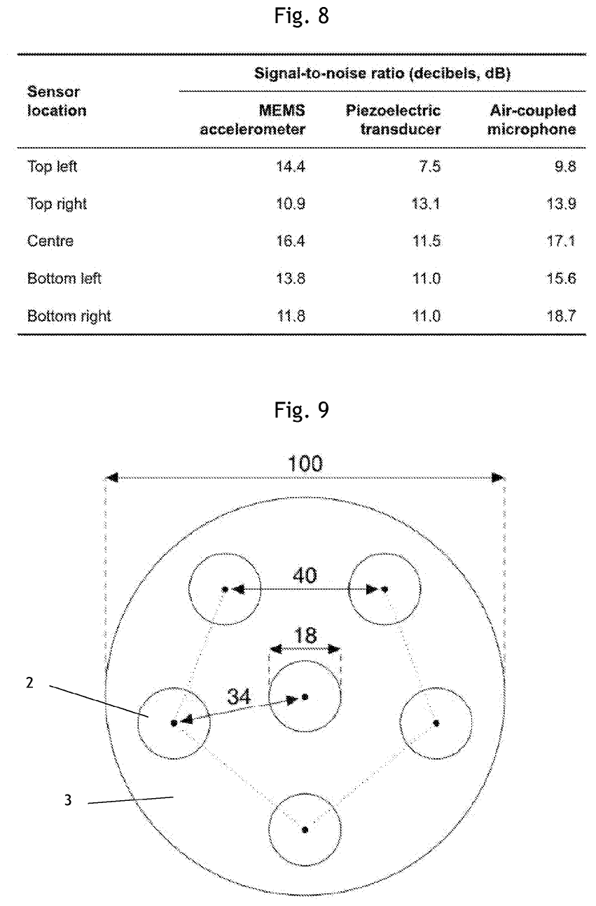

FIG. 7 shows data obtained in the same way as that of FIG. 6, but for devices each having different types of sensors, namely a) MEMS accelerometer, b) piezoelectric transducer, c) air-coupled microphone. FIG. 8 shows the signal to noise ratio obtained for different types of sensors.

It should be understood that variations of the above described examples are possible in light of the above teachings, without departing from the spirit or scope of the disclosure.

1. A data collection device for monitoring a physiological state of a subject, comprising:
a plurality of sensors, each of the plurality of sensors being configured to obtain a biophysical signal from the subject;
a flexible substrate configured to be placed against the subject by a user and configured to flex to substantially conform with the form of the subject, in use;
wherein the plurality of sensors are connected to the substrate and arranged such that each sensor is configured to move relative to each other sensor to enable the substrate to flex to substantially conform to the form of the subject.

2. The data collection device of claim 1, wherein the device is a handheld device and/or the device has a maximum width of no more than 15 cm.

3. (canceled)

4. The device of claim 1, wherein the plurality of sensors are arranged in two dimensions.

5. The device of claim 1, wherein the plurality of sensors comprises a central sensor surrounded by a plurality of further sensors.

6. The device of claim 1, wherein the plurality of sensors are arranged in an arrangement that has bilateral and/or rotational symmetry.

7. The device of claim 1, wherein, adjacent sensors of the plurality of sensors are at least 2 cm apart and/or adjacent sensors of the plurality of sensors are at least 2.5r apart, where r is the radius of the sensor, or half the width of the sensor at its widest point.

8. (canceled)

9. The device of claim 1, wherein adjacent sensors of the plurality of sensors are arranged no more than 5 cm apart and/or sensors of the plurality of sensors that are furthest apart are arranged at least 5 cm apart, and/or sensors of the plurality of sensors that are furthest apart are arranged no more than 15 cm apart.

10.-11. (canceled)

12. The device of claim 1, wherein the plurality of sensors are substantially spread across the flexible substrate.

13. The device of claim 1, wherein the flexible substrate is formed from an elastomeric material, and/or the flexible substrate is formed from silicone, and/or the plurality of sensors are connected to each other by flexible connectors.

14.-15. (canceled)

16. The device of claim 1, wherein each sensor of the plurality of sensors is configured to obtain a vibration signal, and/or wherein each sensor of the plurality of sensors is the same.

17. (canceled)

18. The device of claim 1, wherein the biophysical signal is a vibration signal from the heart of the subject.

19. The device of claim 1, wherein each of the plurality of sensors is configured to obtain a signal simultaneously.

20. A system for monitoring the physiological state of a subject, the system comprising:
the device of claim 1;
one or more processors configured to process the biophysical signals obtained by the plurality of sensors in order to determine the physiological state of a subject.

21. The system of claim 20, wherein the one or more processors is configured to base the determination of the physiological state of a subject on a subset of the biophysical signals obtained by the sensors, wherein the subset of biophysical signals comprises at least one signal that is of a relatively high quality compared to other biophysical signals not in the subset.

22. (canceled)

23. The system of claim 20, wherein the one or more processors are configured to determine the physiological state of a subject based on biophysical signals obtained when the device is placed against the subject at least two different predefined locations.

24. The system of claim 20, wherein the one or more processors form part of an external device.

25. A computer implemented method of monitoring the physiological state of a subject, the system comprising:
receiving data including one or more biophysical signals obtained by the device of claim 1;
processing the data including the one or more biophysical signals to determine the physiological state of a subject.

26. A method of collecting data for monitoring a physiological state of a subject, the method comprising use of the data collection device of claim 1 to obtain a biophysical signal from a subject.

27. The method of claim 26, comprising placing the flexible substrate of the device against the subject at least one location on the subject to obtain the biophysical data, wherein the at least one location corresponds a location suitable for obtaining a biophysical data corresponding to at least one predetermined part of the subject.

28. (canceled)

29. The method of claim 27, wherein the part is the heart, and/or wherein the part is at least one of the tricuspid valve and the mitral valve of the heart, or the part is the lungs, bowel or throat.

30.-31. (canceled)
